(19) United States
(12) Patent Application Publication
(10) Pub. No.: US 2020/0107820 A1
(43) Pub. Date: Apr. 9, 2020

(54) DEVICES FOR PREGNANCY DETECTION AND CORRESPONDING METHODS THEREOF

(71) Applicant: INSTLOO PRIVATE LIMITED, Noida (IN)

(72) Inventor: Poonam Gupta, Noida (IN)

(21) Appl. No.: 16/595,314

(22) Filed: Oct. 7, 2019

(30) Foreign Application Priority Data
Oct. 5, 2018 (IN) 201811037875

(51) Int. Cl. A61B 10/00; G01N 33/68; G01N 33/76

(52) U.S. Cl. CPC A61B 10/007; G01N 2333/59; G01N 33/76; G01N 33/689

(57) ABSTRACT

There is disclosed a device for pregnancy detection is provided, the device comprising: a central main body comprising a first end and a second end; a pregnancy detection element that is mounted at an edge of the second opening, and an upper longitudinal crease and a lower longitudinal crease, wherein the device is opened by applying simultaneous pressure to the upper longitudinal crease and the lower longitudinal crease, wherein, when in operation, the first opening of the first end is configured to receive urine from a female user while the female user is in a standing position and enables the urine to pass through the pregnancy detection element, and wherein, when the urine comes in contact with the pregnancy detection element, the pregnancy detection element immunologically detects human chorionic gonadotropin (hCG) present in the urine of the female user and indicates positive pregnancy result if the female user is pregnant.

### CROSS-REFERENCE TO RELATED APPLICATIONS

This application claims priority to Indian patent application no. 201811037875 filed on Oct. 5, 2018, the complete disclosure of which, in its entirely, is herein incorporated by reference.

### BACKGROUND

### Technical Field

The embodiments herein generally relate to a pregnancy detection device, and, more particularly, to devices for pregnancy detection, and corresponding methods thereof.

### Description of the Related Art

Generally, after fertilization, the placenta soon produces a Human Chorionic Gonadotropin (hCG), which is a glycoprotein. This hormone has the crucial role of maintaining steroid production by the corpus luteum in early pregnancy, and ensures that pregnancy progresses unabated. Thus, the detection of hCG has been utilized as a marker for pregnancy in women. Accordingly, measurement of hCG in blood or urine has been the basis of prior and current pregnancy tests or devices.

Most pregnancy tests are designed to indicate whether a person's urine or blood contains a hormone called human chorionic gonadotropin (hCG). This hormone is produced after a fertilized egg attaches to the wall of a woman's uterus. In non-pregnant women, hCG levels are normally undetectable. If a woman is pregnant, however, levels of hCG continue to rise rapidly, doubling every two to three days.

If a woman is pregnant, the hCG urine test can usually detect this hormone in her urine about 10 days after she has first missed her menstruation cycle. This is when the fertilized egg attaches to the uterine wall. During the first 8 to 10 weeks of pregnancy, hCG levels normally increase very rapidly. These levels reach their peak at about the 10th week of pregnancy, and then they gradually decline until delivery. This type of urine test is commonly sold in kits that users can use at home. It's often is referred to as a home pregnancy test. The hCG urine test confirms pregnancy about one to two weeks after your missed period. This is a qualitative test, which means that it will tell you whether or not it detects the hCG hormone in your urine. It's not intended to reveal specific levels of the hormone. The presence of hCG in a female's urine is considered a positive sign of pregnancy.

Particularly, there are a few home pregnancy tests that can help a woman detect pregnancy at early stage, like, Prega News Pregnancy Test Kit, Velocit Pregnancy Kit, Clear View Pregnancy Test Kit, I-Can Pregnancy Test Kit, Pregcolor Test Card, Pregtest Card, Acutest by Cadila and Velocit Eazy.

However, these existing pregnancy test kits which are available in the market are either in the form of a cassette with dropper or strip with a cup in which expecting women need to store the urine and then perform the pregnancy test. The entire process is unhygienic and messy as the user will come into contact which their urine.

Moreover, the entire process of present pregnancy testing using inline test kits is unhygienic, messy, inconvenient and of discomfort, as the user will need to sit down on the toilet seat, even if she is in a public place and using a public restroom which is not hygienic, store her urine in a dropper or a cup during which, the user comes in contact with their liquid waste (Urine).

Accordingly, there remains a need in the art for innovative, novel, efficient devices for pregnancy detection, and corresponding methods thereof.

### SUMMARY

In accordance with an embodiment of the present disclosure, a device for pregnancy detection includes, a central main body including a first end having a first opening and a second end having a second opening, an upper longitudinal crease and a lower longitudinal crease, and, a pregnancy detection element mounted at an edge of the second opening.

In an aspect of the present disclosure, a device for pregnancy detection is provided, the device comprising: a central main body comprising a first end and a second end, wherein the first end comprising a first opening and the second end comprising a second opening positioned opposite to the first opening, a pregnancy detection element that is mounted at an edge of the second opening; and an upper longitudinal crease and a lower longitudinal crease, wherein the upper longitudinal crease and the lower longitudinal crease are folded to have compact flattened packaging of the device, wherein the device is opened by applying simultaneous pressure to the upper longitudinal crease and the lower longitudinal crease, wherein, when in operation, the first opening of the first end is configured to receive urine from a female user while the female user is in a standing position and enables the urine to pass through the pregnancy detection element at the edge of the second opening, and wherein, when the urine comes in contact with the pregnancy detection element, the pregnancy detection element immunologically detects human chorionic gonadotropin (hCG) present in the urine of the female user and indicates a positive pregnancy result if the female user is pregnant.

In an embodiment, the pregnancy detection element is coated with a plurality of human chorionic gonadotropin (hCG) antibodies for detecting the human chorionic gonadotropin (hCG) present in the urine of the female user. In another embodiment, the pregnancy detection element is capable of testing and indicating the positive pregnancy result, or a negative pregnancy result, or a failed result.

In yet another embodiment, the pregnancy detection element operates by immunologically detecting a plurality of morphological differences between the human chorionic gonadotropin (hCG) and modified forms thereof, which are secreted into the urine of a pregnant female. In yet another embodiment, the first opening of the first end is configured to enable the female user to urinate while in standing position by slightly tilting (bending) her hips in a manner such that the device is tilted downwards.

In yet another embodiment, the pregnancy detection element tests and starts indicating the positive pregnancy result, or the negative pregnancy result or the failed result within 15 seconds of coming in contact with the urine of the female user.

In yet another embodiment, the device is made from a suitable material selected from a group comprising a water-absorbent paper, a cardboard, a water dissolvable plastic-like film, and the like.

In yet another embodiment, the device is biodegradable and environment friendly after being used by the female user. In yet another embodiment, the second opening of the second end is configured to discharge urine from the female user after the urine has come in contact with the pregnancy detection element. In yet another embodiment, a perimeter of the first opening is greater than a perimeter of the second opening.

In yet another embodiment, the first opening comprises a plurality of walls defined through the upper longitudinal crease and the lower longitudinal crease, wherein the plurality of walls comprises inner surfaces coated with a thin, moisture-resistant coating for preventing the urine from passing through the plurality of walls. In yet another embodiment, the device is capable of conducting a plurality of urine related tests, but not limited to, a Urine Tract Infection (UTI) test, and the like.

In another aspect of the present disclosure, a method for pregnancy detection for detecting pregnancy using urine from the female is provided, the method comprising the steps (a) providing a pregnancy detection device to a female user, (b) receiving urine from the female user into a first opening of a first end of the pregnancy detection device, (c) flowing urine from the female user from the first opening towards a pregnancy detection element mounted at an edge of a second opening of a second end of the pregnancy detection device, (d) testing and start indicating a positive pregnancy result, or a negative pregnancy result, or a failed result by the pregnancy detection element within 15 seconds of coming in contact with urine of the female user, and (e) discharging urine from the female user via the second opening after the urine has come in contact with the pregnancy detection element.

In yet another aspect of the present disclosure, a device for pregnancy detection is provided, the device comprising: a central main body comprising a first end and a second end, wherein the first end comprising a first opening and the second end comprising a second opening positioned opposite to the first opening, wherein a perimeter of the first opening is greater than a perimeter of the second opening; a pregnancy detection element that is mounted on an edge of the second opening; and an upper longitudinal crease and a lower longitudinal crease, wherein the upper longitudinal crease and the lower longitudinal crease are folded to have compact flattened packaging of the device, wherein the first opening comprises a plurality of walls defined through the upper longitudinal crease and the lower longitudinal crease, wherein the plurality of walls comprises inner surfaces coated with a thin, moisture-resistant coating for preventing urine from passing through the plurality of walls, wherein the device is opened by applying simultaneous pressure to the upper longitudinal crease and the lower longitudinal crease, wherein, when in operation, the first opening of the first end is configured to receive the urine from a female user while the female user is in a standing position and enables the urine to pass through the pregnancy detection element at the edge of the second opening, wherein, when the urine comes in contact with the pregnancy detection element, the pregnancy detection element immunologically detects human chorionic gonadotropin (hCG) present in the urine of the female user and indicates a positive pregnancy result if the female user is pregnant.

In an embodiment, the pregnancy detection element is coated with a plurality of human chorionic gonadotropin (hCG) antibodies for detecting the human chorionic gonadotropin (hCG) present in the urine of the female user. In another embodiment, the second opening of second end is configured to discharge the urine from the female user after the urine has come in contact with the pregnancy detection element.

In yet another embodiment, the pregnancy detection element tests and starts indicating the positive pregnancy result, or a negative pregnancy result or a failed result within 15 seconds of coming in contact with the urine of the female user. In yet another embodiment, the device is biodegradable and environment friendly after being used by the female user. In yet another embodiment, the device is made from a suitable material selected from a group comprising a water-absorbent paper, a cardboard, a water dissolvable plastic-like film, and the like.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawing, in which:

FIG. 1. illustrates a side view of a device for pregnancy detection, according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

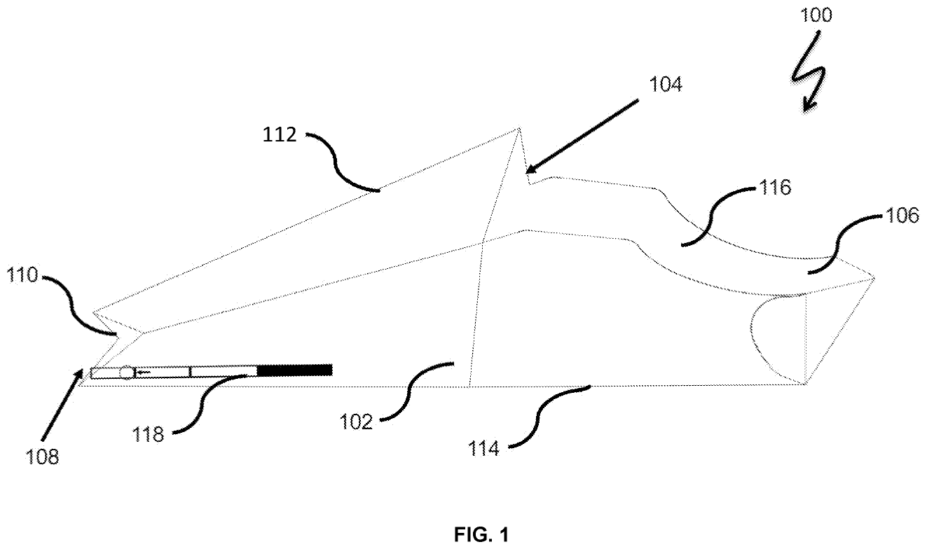

Various embodiments of the present disclosure are disclosed herein below, which relate to devices for pregnancy detection, and corresponding methods thereof. The embodiments of the present disclosure are aimed at providing a portable pregnancy test kit that is suitable for home use, in other words, a modern pregnancy device that replaces the use of a dropper or cup. In one embodiment, the present disclosure discloses a Biotech Fast-Moving Consumer Goods (FMCG) Product, which is a revolutionary product designed for female users to test their pregnancy without storing their urine or collecting their urine in any of the dropper/cup. Referring now to the drawing FIG. 1, where similar reference characters denote corresponding features consistently throughout the FIGURES, there are shown preferred embodiments.

Those of ordinary skills in the art will appreciate that embodiments of the present disclosure disclose a modern pregnancy device that replaces the traditional way to test the pregnancy, which is a very messy process. The device provides the comfort to expecting women to check their pregnancy with ease, speed and hygiene. Essentially, the device as disclosed herein below is a funnel or a vessel or any other suitable shape, which peed into, indicates the pregnancy status. It does not require any dropper or cup to store urine and test the pregnancy. The entire process is hygienic, and user does not come in contact with urine and need not sit on the toilet.

In other embodiments, the present disclosure comprises a device for selectively or preferentially detecting hyper glycosylated human chorionic gonadotropin (hCG-H), in the woman's liquid body waste (Urine) without causing the user to come into contact with the liquid waste.

FIG. 1. illustrates a side view of a device 100 for pregnancy detection, according to an embodiment herein. The device 100 for pregnancy detection includes, a central main body 102 including a first end 104 and a second end 108. In use, the first end 104 includes a first opening 106 and the second end 108 includes a second opening 110 positioned opposite to the first opening 106.

In accordance with an embodiment of the present disclosure, the device 100 further includes an upper longitudinal crease 112 and a lower longitudinal crease 114. In use, the upper longitudinal crease 112 and the lower longitudinal crease 114 are foldable and folded to have compact flattened packaging of the device 100.

In accordance with an embodiment of the present disclosure, a perimeter of the first opening 106 is greater than a perimeter of the second opening 110. Also, the first opening 106 includes a plurality of walls 116 that are defined through the upper longitudinal crease 112 and the lower longitudinal crease 114, and the plurality of walls 116 includes inner surfaces coated with a thin, moisture-resistant coating for preventing urine from passing through the walls. Additionally, the device 100 is opened by applying simultaneous pressure to the upper longitudinal crease 112 and the lower longitudinal crease 114.

In accordance with an embodiment of the present disclosure, the device 100 further includes a pregnancy detection element 118 that is mounted at an edge of the second opening 110. In use, the pregnancy detection element 118 tests the urine and indicates a positive pregnancy result, or a negative pregnancy result, or a failed result. In operation, the pregnancy detection element 118 operates by immunologically detecting a plurality of morphological differences between human chorionic gonadotropin (hCG) and modified forms thereof, which are secreted into the body fluid (e.g. urine) of a pregnant female.

In accordance with an embodiment of the present disclosure, the first opening 106 of the first end 104 is configured to receive urine from a female user. In use, the first opening 106 of the first end 104 is configured to allow the female user to urinate while in standing position by slightly tilting (bending) her hips in a manner such that the device 100 is tilted downwards.

In accordance with an embodiment of the present disclosure, the pregnancy detection element 118 tests and starts indicating the positive pregnancy result, or the negative pregnancy result or the failed result within 15 seconds of coming in contact with urine of the female user.

In accordance with an embodiment of the present disclosure, the device 100 is made from a suitable material selected from a group including water-absorbent paper, cardboard, water dissolvable plastic-like film, and the like. In use, the device 100 is biodegradable and environment friendly after being used by the female user.

In accordance with an embodiment of the present disclosure, the second opening 110 of the second end 108 is configured to discharge the urine from the female user after the urine has come in contact with the pregnancy detection element.

Those of ordinary skills in the art will appreciate that by way of alternate embodiments, the device 100 is capable of conducting one or more urine related tests as well, such as, for example, but not limited to, a UTI (Urine Tract Infection) test, an ovulation or a fertility test, and the like. In use, such alternate embodiments shall include suitable replacements for the pregnancy detection element 118, as per the specific requirements

In accordance with an embodiment of the present disclosure, a method for pregnancy detection includes the steps of: providing a pregnancy detection device to a female user; receiving urine from the female user into a first opening of a first end of the pregnancy detection device; flowing urine from the female user from the first opening towards a pregnancy detection element mounted at an edge of a second opening of a second end of the pregnancy detection device; testing and start indicating a positive pregnancy result, or a negative pregnancy result, or a failed result by the pregnancy detection element within 15 seconds of coming in contact with urine of the female user; and, discharging urine from the female user via the second opening after the urine has come in contact with the pregnancy detection element.

Therefore, as may be seen, various embodiments of the present disclosure as disclosed hereinabove provide significant advantages over prior art, such as, for example, but not limited to, providing a novel and inventive pregnancy test kit which can be used while standing and simply passing the liquid body waste in the product without coming into contact with the urine. Also, this present disclosure keeps the health care of all expecting woman in mind and is therefore very hygienic. Generally, embodiments of the instant disclosure will help the user to discard unhygienic process and provide the ease of use to test their pregnancy in coming era. The embodiments herein provide the great comfort to user to stand and test their pregnancy which none other product is providing in the market. The embodiments herein are also bio-degradable in nature, and, in simple words, it's a step ahead towards the ease of use.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” ‘having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While there has been shown and described the preferred embodiment of the instant disclosure it is to be appreciated that the present disclosure may be embodied otherwise than is herein specifically shown and described and that, within said embodiment, certain changes may be made in the form and arrangement of the parts without departing from the underlying ideas or principles of this present disclosure as set forth in the Claims appended herewith. Therefore, the appended claims are to be construed to cover all equivalents falling within the true scope and spirit of the present disclosure.

What is claimed is:

1. A device for pregnancy detection, said device comprising:
a central main body comprising a first end and a second end, wherein said first end comprising a first opening and said second end comprising a second opening positioned opposite to said first opening;
a pregnancy detection element that is mounted at an edge of said second opening; and
an upper longitudinal crease and a lower longitudinal crease, wherein said upper longitudinal crease and said lower longitudinal crease are folded to have compact flattened packaging of said device;
wherein said device is opened by applying simultaneous pressure to said upper longitudinal crease and said lower longitudinal crease;
wherein, when in operation, said first opening of the said first end is configured to receive urine from a female user while said female user is in a standing position and enables the urine to pass through said pregnancy detection element at the edge of said second opening, and wherein, when the urine comes in contact with said pregnancy detection element, said pregnancy detection element immunologically detects human chorionic gonadotropin (hCG) present in the urine of the female user and indicates a positive pregnancy result if said female user is pregnant.

2. The device according to claim 1, wherein said pregnancy detection element is coated with a plurality of human chorionic gonadotropin (hCG) antibodies for detecting said human chorionic gonadotropin (hCG) present in the urine of the female user.

3. The device according to claim 2, wherein said pregnancy detection element is capable of testing and indicating said positive pregnancy result, or a negative pregnancy result, or a failed result.

4. The device according to claim 1, wherein said pregnancy detection element operates by immunologically detecting a plurality of morphological differences between the human chorionic gonadotropin (hCG) and modified forms thereof, which are secreted into the urine of a pregnant female.

5. The device according to claim 1, wherein said first opening of said first end is configured to enable said female user to urinate while in standing position by slightly tilting (bending) her hips in a manner such that said device is tilted downwards.

6. The device according to claim 3, wherein said pregnancy detection element is capable of testing and starts indicating said positive pregnancy result, or said negative pregnancy result or said failed result within 15 seconds of coming in contact with the urine of said female user.

7. The device according to claim 1, wherein said device is made from a suitable material selected from a group comprising a water-absorbent paper, a cardboard, a water dissolvable plastic-like film, and the like.

8. The device according to claim 1, wherein said device is biodegradable and environment friendly after being used by said female user.

9. The device according to claim 1, wherein said second opening of said second end is configured to discharge the urine from said female user after the urine has come in contact with said pregnancy detection element.

10. The device as claimed in claim 1, wherein a perimeter of said first opening is greater than a perimeter of said second opening.

11. The device as claimed in claim 1, wherein said first opening comprises a plurality of walls defined through said upper longitudinal crease and said lower longitudinal crease, wherein said plurality of walls comprises inner surfaces coated with a thin, moisture-resistant coating for preventing the urine from passing through said plurality of walls.

12. The device as claimed in claim 1, wherein said device is capable of conducting a plurality of urine related tests, but not limited to, a Urine Tract Infection (UTI) test, an ovulation or a fertility test, and the like.

13. A method for pregnancy detection for detecting pregnancy using urine from the said female, said method comprising the steps of:
providing a pregnancy detection device to a female user;
receiving urine from said female user into a first opening of a first end of said pregnancy detection device;
flowing urine from said female user from said first opening towards a pregnancy detection element mounted at an edge of a second opening of a second end of said pregnancy detection device;
testing and start indicating a positive pregnancy result, or a negative pregnancy result, or a failed result by said pregnancy detection element within 15 seconds of coming in contact with urine of said female user; and,
discharging urine from said female user via said second opening after said urine has come in contact with said pregnancy detection element.

14. A device for pregnancy detection, said device comprising:
a central main body comprising a first end and a second end, wherein said first end comprising a first opening and said second end comprising a second opening positioned opposite to said first opening, wherein a perimeter of said first opening is greater than a perimeter of said second opening;
a pregnancy detection element that is mounted at an edge of said second opening; and
an upper longitudinal crease and a lower longitudinal crease, wherein said upper longitudinal crease and said lower longitudinal crease are folded to have compact flattened packaging of said device;
wherein said first opening comprises a plurality of walls defined through said upper longitudinal crease and said lower longitudinal crease, wherein said plurality of walls comprises inner surfaces coated with a thin, moisture-resistant coating for preventing urine from passing through said plurality of walls; and,
wherein said device is opened by applying simultaneous pressure to said upper longitudinal crease and said lower longitudinal crease,
wherein, when in operation, said first opening of the said first end is configured to receive the urine from a female user while said female user is in a standing position and enables the urine to pass through said pregnancy detection element at the edge of said second opening, wherein, when the urine comes in contact with said pregnancy detection element, said pregnancy detection element immunologically detects human chorionic gonadotropin (hCG) present in the urine of the female user and indicates a positive pregnancy result if said female user is pregnant.

15. The device according to claim 14, wherein said pregnancy detection element is coated with a plurality of human chorionic gonadotropin (hCG) antibodies for detecting said human chorionic gonadotropin (hCG) present in the urine of the female user.

16. The device according to claim 14, wherein said second opening of said second end is configured to discharge the urine from said female user after the urine has come in contact with said pregnancy detection element.

17. The device according to claim 14, wherein said pregnancy detection element is capable of testing and start indicating said positive pregnancy result, or a negative pregnancy result or a failed result within 15 seconds of coming in contact with the urine of said female user.

18. The device according to claim 14, wherein said device is biodegradable and environment friendly after being used by said female user.

19. The device according to claim 14, wherein said device is made from a suitable material selected from a group comprising a water-absorbent paper, a cardboard, a water dissolvable plastic-like film, and the like.
